(19) United States
(12) Patent Application Publication
(10) Pub. No.: US 2007/0032865 A1
(43) Pub. Date: Feb. 8, 2007

(54) Prosthesis having a coating and systems and methods of making the same

(76) Inventors: David R. Otis, Corvallis, OR (US); Hirdey Bhathal, San Diego, CA (US); Isaac Farr, Aguadilla, PR (US); N.K. Peter Samuel, Corvallis, OR (US); Bradley Bower, Junction City, OR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/197,855

(22) Filed: Aug. 5, 2005

(51) Int. Cl. A61F 2/06

(52) U.S. Cl. 623/1.46; 623/1.15; 623/926; 427/2.25; 118/696

(57) ABSTRACT

Implantable medical devices such as stents, methods of making medical devices, and systems for making medical devices are disclosed herein. In one exemplary embodiment, medical device includes a stent that is coated at least on a portion of one surface by an ink-jet device.

### BACKGROUND

The present disclosure relates to prostheses and systems and methods of making the prostheses.

A variety of stent coatings and compositions have been proposed for the prevention and treatment of, for example, injury causing intimal thickening. The coatings may be capable themselves of reducing the stimulus the stent provides to the injured lumen wall, thus reducing the tendency towards thrombosis or restenosis. Alternately, the coating may deliver a pharmaceutical/therapeutic agent or drug to the lumen that reduces smooth muscle tissue proliferation or restenosis. The mechanism for delivery of the agent is either through (1) diffusion of the agent through either a bulk polymer, (2) transfer through pores that are created in the polymer structure, (3) erosion of a biodegradable coating, or (4) surface release from the polymer.

Both bioabsorbable and biostable compositions have been reported as coatings for stents. They generally have been polymeric coatings that either encapsulate a pharmaceutical/therapeutic agent or drug, e.g., rapamycin, taxol, etc., or bind such an agent to the surface, e.g., heparin-coated stents. These coatings are applied to the stent in a number of ways, including, though not limited to, dip, spray, or spin coating processes.

The practice of coating implantable medical devices with a synthetic or biological active or inactive agent is known. Numerous processes have been proposed for the application of such a coating. For example, soaking or dipping the implantable device in a bath of liquid medication has been suggested, as well as soaking in an agitated bath. Devices have been disclosed for introducing heat and/or ultrasonic energy in conjunction with the medicated bath. Alternatively, the medication can be sprayed on a medical device.

Dip-coating, with subsequent airflow through the core of the stent, does not allow the thickness of the coating to be controlled and does not allow for parts of a stent to be preferentially coated. Additionally, surface effects from this process can result in “bridging” of the coating over open areas between struts of the stent. Conventional spray-coating has the problems of inexact material placement, particularly when more than one coating is desired. Overspray onto the stent is possible, resulting in wasting of the coating material.

It is known to use inkjet technology to apply a liquid to selected portion of a surface. In the paper “Applications of Ink-Jet Printing Technology to BioMEMS and Microfluidic Systems,” presented at the SPIE Conference on Microfluidics and BioMEMS, October, 01, the authors, Patrick Cooley, David Wallace, and Bogdan Antohe provide a fairly detailed description of Ink-Jet technology and the range of its medically-related applications.

One related device uses a moveable two-dimensional array of nozzles to deposit a plurality of different liquid reagents into receiving chambers. Other systems which use ink-jet applicators that apply the coating with a “freestyle” procedure. The freestyle points are determined by a preprogrammed user selected pattern that is unique to the particular shape or contour for the type of prosthesis and the desired coating to be achieved, much like a vector based printing approach. The ink-jet nozzle or prosthesis moves in three-dimensions with the aid of a motion control system. The motion control system enables the ink-jet nozzle to move over the portions of the prosthesis to be coated. Alternatively, a real-time picture can be taken with a camera to determine the position of the ink-jet nozzle in relation to the prosthesis. Based upon the feedback of nozzle location, the ink-jet applicator can be controlled by activating the spray, moving the ink-jet nozzle, and/or moving the prosthesis to adjust to the pattern to better conform with the actual prosthesis.

It would be advantageous to develop coatings for and methods of coating implantable medical devices that will reduce thrombosis, restenosis, or other adverse reactions, that may include, but do not require, the use of pharmaceutical or therapeutic agents or drugs to achieve such affects, and that possess physical and mechanical properties effective for use in such devices.

### SUMMARY

Briefly described, embodiments of this disclosure prostheses, prosthesis coating systems, and methods of coating a prosthesis. One exemplary prosthesis, among others, includes a coating deposited on the prosthesis with a drop-on-demand device and a capillary groove disposed in at least one surface of the prosthesis, the groove being configured to retain and guide the deposition of, at least some portion of the coating.

One exemplary method, among others, of coating a prosthesis includes the steps of: depositing a plurality of droplets of a material on a prosthesis, wherein the droplets are deposited by an ink-jet device, and wherein each of the droplets has a volume of at least about 5 picoliters.

One exemplary prosthesis coating system includes a prosthesis positioned on a rotatable chuck; an ink-jet device in proximity to the prosthesis, whereby droplets of a material from the ink-jet device can coat at least a portion of the prosthesis; and an optical micrometer configured to measure material being deposited on the rotating prosthesis by the ink-jet device, wherein the prosthesis is positioned between a transmitter and a receiver in the optical micrometer.

Other prostheses, prosthesis coating systems, and methods of coating a prosthesis are described in more detail below.

### BRIEF DESCRIPTION OF THE DRAWINGS

Many aspects of this disclosure can be better understood with reference to the following drawings. The components in the drawings are not necessarily to scale. Moreover, in the drawings, like reference numerals designate corresponding parts throughout the several views.

FIG. 1 is an illustrative representation showing an elevational view of an embodiment of the stent coating apparatus.

FIG. 2 is an illustrative representation showing an elevational view of an embodiment of the disclosed stent coating apparatus.

FIG. 3 is an illustrative representation of an enlarged view of an embodiment of the disclosed stent.

FIG. 4 is an illustrative representation of an enlarged view of an embodiment of the disclosed stent.

FIGS. 5A and 5B are illustrative representations of an enlarged view of embodiment of the disclosed stent, (A) before deployment in a patient; and (B) after deployment in a patient.

FIG. 6 depicts a micrograph of material layers deposited by a drop-on-demand device applied to a stainless steel tube, at a magnification level indicated by the 500-micron-long bar.

### DETAILED DESCRIPTION

Definitions

The term “prosthesis” refers to any one of many medical coating applications including but not limited to coronary stents, peripheral vascular stents; abdominal aortic aneurysm (AAA) devices, biliary stents and catheters, TIPS catheters and stents, vena cava filters, vascular filters and distal support devices and emboli filter/entrapment aids, vascular grafts and stent grafts, gastro enteral tubes/stents, gastra enteral and vascular anastomotic devices, urinary catheters and stents, surgical and wound drainings, radioactive needles and other indwelling metal implants, bronchial tubes and stents, vascular coils, vascular protection devices, tissue and mechanical prosthetic heart valves and rings, arterial-venous shunts, AV access grafts, surgical tampons, dental implants, CSF shunts, pacemaker electrodes and leads, suture material, wound healing, tissue closure devices including wires, staplers, surgical clips etc., IUDs and associated pregnancy control devices, ocular implants, timponoplasty implants, hearing aids including cochlear implants, implantable pumps (like insulin pumps), implantable cameras and other diagnostic devices, drug delivery capsules, left ventricular assist devices (LVADs) and other implantable heart support and vascular systems, indwelling vascular access catheters and associated devices (like ports), maxilo facial implants, orthopedic implants (joint replacement, trauma management and spine surgery devices), implantable devices for plastic and cosmetic surgery, implantable meshes (such as for hernia or for uro-vaginal repair, brain disorders, and gastrointestinal ailments).

The term “ink-jet” refers to any release of a drop or number of drops. Ink-jet technology encompasses both drop-on-demand technologies and continuous ink-jet delivery technology.

The term “drop-on-demand” refers to any ejection of essentially a drop of a desired quantity of coating material. “Essentially a drop” includes the drop itself and any number of satellite drops. One example of “drop-on-demand” is the piezo drop-on-demand technology such as that manufactured by Ink Jet Technology, Inc. of San Jose, Calif. which provides applicators for a wide variety of coating applications. With a built in heater and high temperature operating potential, piezo drop-on-demand applicators are compatible with a wide variety of coating materials.

Another example of drop-on-demand dispense technology is thermal DOD technology, also known as thermal ink-jet (or TIJ) technology. Drop-on-demand delivery can be performed “on-the-fly,” e.g., synchronous or close to synchronous, and/or simultaneous or close to simultaneous. Unlike freestyle movement which requires stopping for validation of preceding and subsequent movement with relation to the prosthesis, on-the-fly continues to next movement without validation step.

“Continuous ink-jet” also refers to jetting when a sequence of drops is released. Further information about ink-jet technology can be found in the reference “Ink-jet Technology and Product Development” by Stephen F. Pond, Ph.D., Torrey Pines Research, 2000.

The term “detector” or “detecting” refers to any device or method which uses energy, such as magnetic, electrical, heat, light, etc. to determine whether a target at a desired location on the prosthesis has been located and signals the applicator to drop-on-demand or marks the location as one to be coated. The detector does not determine the location of the applicator relative to the target to provide feedback for positioning the applicator. The detector determines the points on the coordinate table for desired locations on the prosthesis by providing signals for the applicator controller that are immediately used or stored as coordinate tables. Examples of detectors are light sensitive devices such as CCD area cameras, CCD line cameras, high-resolution CMOS area cameras, or devices that can capture light reflected or transmitted by the prosthesis, and electrically sensitive devices such as capacitance detectors.

The term “applicator” or “applying” refers to any configuration, apparatus, or method for positioning a coating material to a surface from a reservoir such as a point source including but not limited to a nozzle, a dispenser, or tip, or a multipoint source. An example of an applicator is a drop-on-demand ink-jet.

The term “on-the-fly” refers to translation and drop-on-demand delivery that is synchronous or close to synchronous, and/or simultaneous or close to simultaneous. Unlike freestyle movement which requires stopping for validation of preceding and subsequent movement with relation to the prosthesis, on-the-fly continues to next movement without validation step.

It is noted that, as used in this specification and the appended claims, the singular forms “a,” “an,” and “the” include plural referents unless expressly and unequivocally limited to one referent. Thus for example, reference to “an applicator” includes two or more applicators, but “n is an integer from 1 to 60” means that n is one integer because that is limited to one integer. Further, the term “plurality” should be interpreted to include both more than one, and a multitude.

Also noted that as used herein, the term “polymer” is meant to refer to oligomers, homopolymers, and copolymers. The term “therapeutic agent” is meant to refer to drugs, therapeutic materials, diagnostic materials, inerts, active ingredients, and inactive ingredients.

For the purposes of this specification and appended claims, unless otherwise indicated, all numbers expressing quantities of ingredients or percentages or proportions of other materials, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Drop-on-demand deposition of material onto a prosthesis requires no tooling, or masks or screens, which also means that there is not contact between the deposition apparatus and the prosthesis. Additionally, inkjet deposition on prostheses is data-driven and the printing information can be created from computer-assisted design (CAD) information, and stored digitally.

By way of introduction, the embodiment discussed herein is a device for applying a medical coating or material to a stent. Although the specific embodiments herein are discussed with respect to a stent, one skilled in the art would understand that that any implantable prosthesis can be utilized, as within the definition of prosthesis above. The coating can be, for example, a polymer, a therapeutic agent (e.g., anti-proliferative, anti-coagulant, antiinflammatory, antibacterial, antiviral, and/or antifungal), a polymer/therapeutic agent mixture, a lubricious material, an adhesion promoter, an adhesion discourager (e.g., modified silane coupling agents), a hydrophobic material, a hydrophilic material, and a diffusion barrier that is designed to reduce the rate of diffusion of a material from the prosthesis to the patient. Such coatings should be hardy enough to withstand the physical demands of deployment into a patient without substantial and/or unpredictable cracking and/or delamination. Substantial and/or unpredictable cracking of material before or during deployment of the stent in the patient can cause, for example, clotting or “embolic events.”

A processing unit selectively activates a coating applicator so as to apply the coating to selected portions of the stent. The coating applicator discussed herein is, by non-limiting example, a pressure-pulse actuated drop-ejection system with at least one nozzle. A readily available pressure-pulse actuated drop-ejection system, which is well suited for the present embodiments, is a drop-on-demand (DOD) dispense system. It should be noted, however, that any coating application system that may be selectively activated is within the intentions of the present disclosure such as, for example, piezo DOD, thermal inkjet DOD, acoustic drop ejection, or electrostatic inkjet (or “continuous inkjet”).

The DOD coating system can be used either by itself or in tandem with dipping or spray-coating processes. Multiple materials, and combinations thereof. Real-time coating thickness measurements can be made, and the coating process can be enhanced by both etching channels or grooves in portions of the prosthesis, and the use of electrostatics to guide droplets to the desired location on the prosthesis.

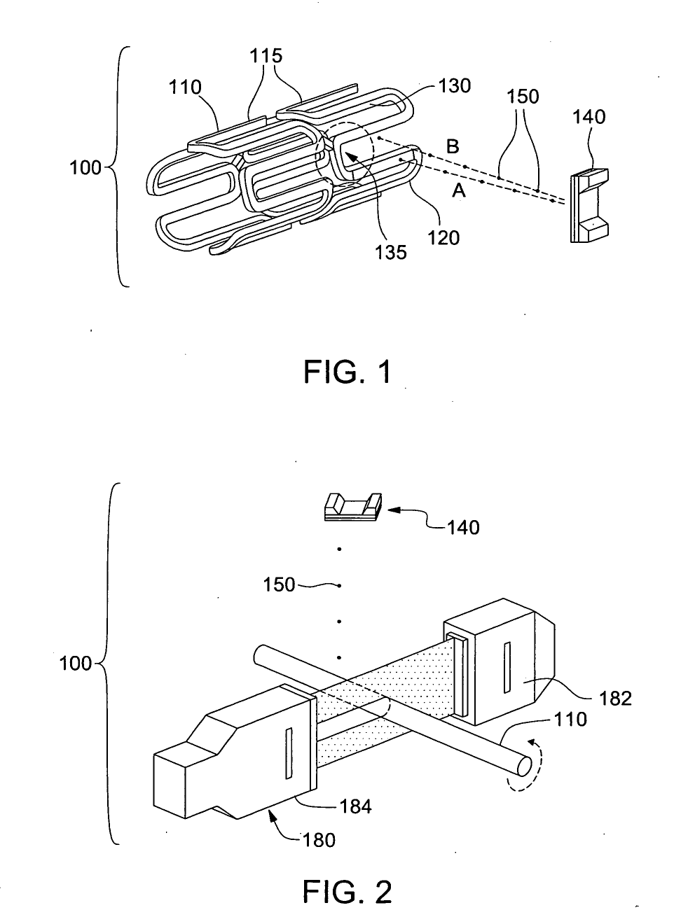

Thus, having generally described the disclosed prosthesis, reference is made to the drawings. Turning now to FIG. 1, shown is a portion of the disclosed prosthesis-coating apparatus 100. The apparatus 100 includes the prosthesis 110 (shown as a stent with struts 115 configured in a sinuous geometry) and a portion of DOD device 140 (showing dispenser only). Droplets 150 of a volume of about 5 picoliters (pl) or larger are directed at the stent strut 115 from the DOD device 140 dispenser. The DOD device 140 is shown to be coating an outside surface 120 on the stent 110 with droplets 150 via the path A. The DOD device 140 is also shown to be coating an inside surface 130 on the stent 110 with droplets 150 via the path B. The curved portion 135 of one of the struts is circled as an “area of maximum strain” 135 (discussed further hereinafter).

In one embodiment, the DOD-to-stent distance is less than or equal to about 1.5 millimeters. If greater DOD-to-stent distance are used, as when depositing droplets 150 on the inside surface 130 of the stent 110, then higher momentum drops (e.g., greater than or equal to about 30 pl and greater than about 8 meters/second (m/s)) can be employed. In this manner the inside surface 130 of the strut 115 on the opposite side of the stent 110 from the DOD device 140 can be coated, for example on a stent with a diameter of about 1-3.5 mm.

Placement accuracy of the droplets on the surface of the stent 110 is about +/−21 microns. Struts 115 can be coated with variable thickness, depending on the number of droplets 150 to be deposited per surface area. Cross-flow and electrostatic effects can be controlled to assure proper droplet trajectory. For example, it has been determined through experimentation that if electrostatic effects are significant, sharp edges on the strut 115 will preferentially receive the droplet 150. In certain applications, where precision placement requirements exceed what the DOD device 140 can accomplish alone, electrostatic effects can be used to steer droplets to sharp edges on the strut 115, where the electrical field is concentrated. Therefore, stents 110 can be employed with sharp edges/features that can help control coating topology, both by electrostatic effects (e.g., where the droplet lands) and/or by capillary effects (e.g., how the liquid flows on the strut once it lands).

Table 1 below illustrates some exemplary settings that can be used for a charged 51-pl droplet fired at 10 meters/second towards a round strut that is 0.7 millimeters (mm) away.

TABLE 1
Stent Coating Example, with electrostatic effect

| Parameter (Units) | Quantity |
|---|---|
| Wire diameter (microns) | 150 |
| Nozzle-to-wire distance (microns) | 1500 |
| y-offset of nozzle (microns) | 75 |
| voltage applied to wire (kV) | 1 |
| initial droplet velocity (m/sec) | 8 |
| drop radius (microns) | 23 |
| charge on droplet (Coulombs) | 0.1 |
| droplet velocity at impact (m/sec) | 9.6 |
| droplet volume (pl) | 51 |
| Weber No. at impact = kinetic | 11.9 |
| energy/surface energy | |
| Re_{droplet} at impact | 29.6 |

Table 2 below illustrates some of exemplary settings that can be used for a droplet, where the electrostatic effects are suppressed.

TABLE 2
Stent Coating Example, with electrostatic effects SUPPRESSED

| Parameter (Units) | Quantity |
|---|---|
| Wire diameter (microns) | 150 |
| Nozzle-to-wire distance (microns) | 1500 |
| y-offset of nozzle (microns) | 75 |
| voltage applied to wire (kV) | 0 |
| initial droplet velocity (m/sec) | 8 |
| drop radius (microns) | 23 |
| charge on droplet (Coulombs) | 0 |
| droplet velocity at impact (m/sec) | 7.8 |
| droplet volume (pl) | 51 |
| Weber No. at impact = kinetic | 7.7 |
| energy/surface energy | |
| Re_{droplet} at impact | 23.8 |

It should be understood that more than one material can be deposited on the stent strut 115, in precise positions. More than one material can be placed using the DOD device 140 alone or in combination with one or more other DOD devices. For example, both the outside surface 120 and the inside surface 130 can be coated with two different DOD dispensers 140 with two different materials. Different coatings can be interlaced to achieve the desired concentration of a drug, making the transition in concentration from one part of the strut 115 to another abrupt or gradual, as desired. The use of multiple DOD dispensers 140 with materials (e.g., therapeutic agents) of different concentrations allow one to modify the concentration gradient (radially, axially, or circumferentially) of therapeutic agent in the coating.

The number of distinct dispensers 140 with different therapeutic agents, polymers, and concentrations is without limit. Use of multiple dispensers with different combinations of materials can be used to form a virtually infinite variety of coatings, with smooth or discrete changes in concentration or thickness, as desired. Stent surfaces 120, 130 can be selectively prepared via application of etchants via the DOD device 140. When applying multiple layers of material with the DOD device 140 (e.g., A followed by B, B followed by C, with A against the stent surface), the layers of coating cannot be dissolved, removed, leached, or otherwise significantly rearranged, as can happen with sequential dip coating processes. For example, if a solvent in dipping bath B significantly dissolves the material applied from dipping bath A, then material A from the stent will leach into the dipping bath B, contaminating the bath over time. With use of the DOD device 140, however, to apply the layers, there may be some interaction between the layers A and B, but layer B will nonetheless be applied in an uncontaminated form.

Additionally, the apparatus 100 can be used to provide coatings on stents 110 of various types of geometries, such as sinuous geometry (shown) or bifurcating stents (not shown). Precise placement of the droplets 150 enabled by the apparatus 100 allows parts of the stent 110 that require no coating or special coatings (e.g., radio-opaque marker areas) to be coated or left bare, as desired.

FIG. 2 depicts another embodiment of the apparatus 100 that includes an optical micrometer 180. In this embodiment, the stent 110 is placed on or in a rotatable chuck, and rotated during deposition of droplets 150 from the DOD device 140. The stent is disposed between a transmitter 182 and a receiver 184 of the optical micrometer 180. The transmitter 182 houses a light transmitter (not shown), while the receiver 184 houses the measuring head that receives the transmitted light and forms an image. An exemplary optical micrometer 180 that can be used in the apparatus 100 includes a high speed LED/CCD optical micrometer, such as LS-7010 series micrometer, Manufactured by and commercially available from Keyence (UK) Ltd. of Milton Keynes, United Kingdom.

The optical micrometer 180 measures film thickness on the stent struts 115. For example, the stent can be rotated about its axis on the chuck to establish a baseline reference prior to deposition of a material on the stent 110. During deposition of droplets 150, the stent 110 can continue to be rotated, while the optical micrometer 180 measures the thickness of the coating being formed from the droplets 150. Measurement accuracy of 0.5 micron can be achieved in this manner. Volatile solvents in the coating are evaporated off before a measurement of “final” coating thickness is made. The evaporation of any solvents in the coating can be facilitated by heating the stent strut, using a high-vapor-pressure solvent, and/or keeping the relative humidity of the solvent in question low.

In this manner, the thickness of the coating formed by the droplets 150 can be controlled to be either conformal or variable, either along its axis or its circumference. The conformal coating is a coating having uniform thickness. The conformal coating thickness can be controlled with the disclosed apparatus 100 such that the coating does not vary in thickness by more than about one micrometer (one micron). Alternatively, the coating can be “stepped” or non-uniformly thick.

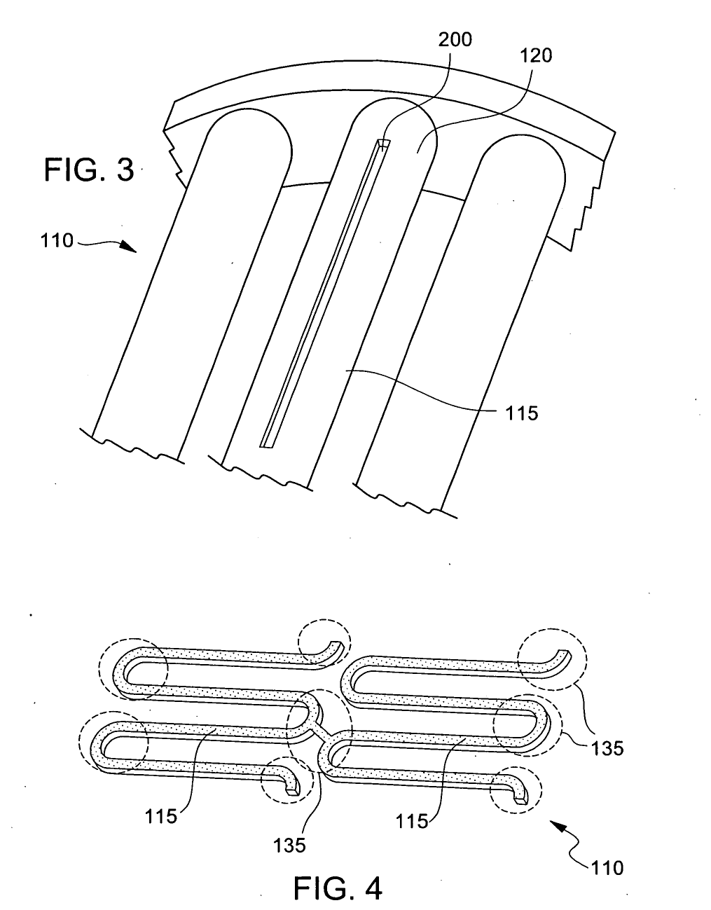

FIG. 3 depicts an enlarged view of an embodiment of the outside surface 120 of a strut 115 of the disclosed stent 110. The strut 115 has a groove 200 formed therein. The groove 200 can be, for example, a capillary-sized groove that holds/retains the material from the droplets 150 that have been applied by the DOD device 140, spray coating, or dipping. The groove 200 can be a simple linear one, such as that shown in FIG. 3, or one of a more complex geometry, for example, a sinuous geometry. The groove 200 can be formed by laser-cutting, etching, or water-jet cutting. The groove 200 can be placed anywhere on the stent 110 that is desired (e.g., outside surface 120, inside surface 130, etc.). The groove 200 serves as a reservoir of material in the stent 110 that can, for example, extend the time period over which a therapeutic agent elutes. Additionally and/or alternatively, the groove 200 functions to keep the material deposited from droplets 150 in a particular location on the stent 110. The edge of the groove 200 sets up a boundary for the contact line, preventing the material, while still wet after dispensing, from advancing or spreading out over the surface of the stent 110. Use of the DOD device 140 to place material in the groove 200 results in more precise placement and filling of the groove 200 than ordinarily obtainable with spraying or dip coating processes.

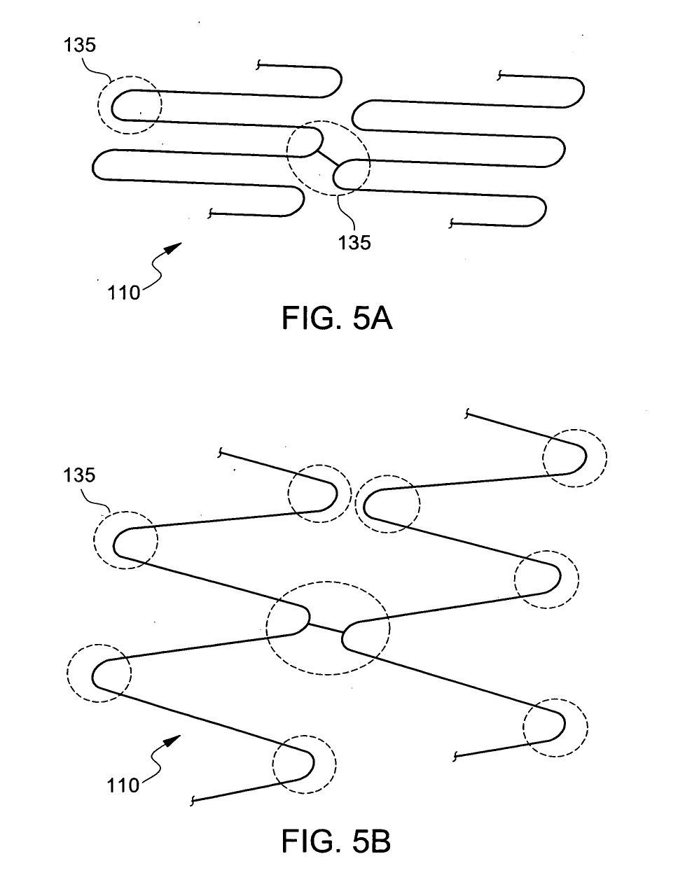

FIGS. 4, 5A, and 5B demonstrate how certain parts 135 of the stent 110 experience more strain than others during deployment. Such a location is termed herein a “maximum strain area” (MSA). As shown in FIG. 4, the MSAs 135 can be curved (or “loop”) areas, or “bridge” areas that connect one strut 115 to another.

If the mechanical properties of the coating and stent struts are identical, then the probability of cracking or delamination on deployment is small. However, in metallic stents, the coating matrix (or “release matrix”) is polymeric, then the mechanical properties of the coating and stent are quite different. Leaving the MSAs uncoated, and preferentially coating only those areas that experience less stress on deployment, as shown in FIGS. 5A and 5B, reduces this risk. FIG. 5A is an exaggerated view of the stent 110 before deployment, with the MSA's 135 encircled. When a stent is expanded, for example, against a vascular wall, the struts 115 undergo significant reshaping and strain, as shown in FIG. 5B. For drug-eluting stents that have struts coated with a layer of a polymer/therapeutic agent mixture, the mixture should substantially adhere to the strut. It is difficult for the mixture to remain attached to MSAs 135. The apparatus 100 allows the MSAs 135 to remain uncoated, thus reducing the odds of coating flake-off or delamination during or after stent expansion. It should be noted that there are many different stent designs, and the disclosed stent embodiment with uncoated MSAs can apply to any stent that experiences spatial variation in strain during deployment, including both self-expanding and balloon-expanded stents.

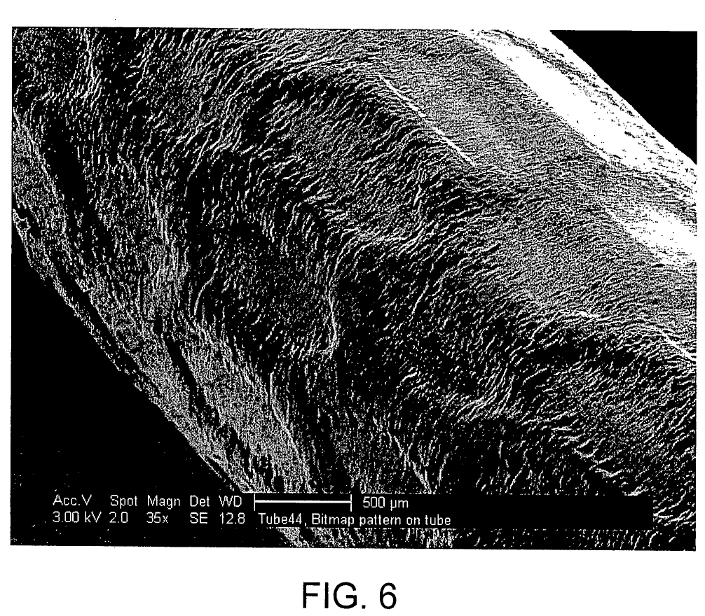

FIG. 6 is a micrograph of a polymeric material pattern deposited by the apparatus 100 applied to a ⅛″ outer diameter 316 stainless steel tube; the 500-micron-long scaling bar is shown. Of note is that the apparatus 100 allows deposition of traces of about 200 microns wide or less, while leaving adjacent areas bare.

Many variations and modifications may be made to the above-described embodiments. All such modifications and variations are intended to be included herein within the scope of this disclosure and protected by the following claims.

The following is claimed:

1. An prosthesis that can implanted into a patient, the prosthesis comprising:
a coating deposited on the prosthesis; and
a capillary groove disposed in at least one surface of the prosthesis, the groove configured to retain and guide the deposition of at least some portion of the coating.

2. The prosthesis of claim 1, wherein the prosthesis is comprised of a wholly or partially biodegradable material.

3. The prosthesis of claim 1, wherein the prosthesis is comprised of a wholly or partially metallic material.

4. The prosthesis of claim 1, wherein the coating comprises multiple layers, wherein each layer is a different material.

5. The prosthesis of claim 1, wherein the coating comprises a material chosen from: a polymer, a therapeutic agent, a polymer/drug mixture, a lubricious material, an adhesion promoter, a hydrophobic material, a hydrophilic material, a diffusion material, and combinations thereof.

6. The prosthesis of claim 1, wherein fluid dispensed from the drop-on-demand device comprises at least one material to control drop ejection and placement, wherein the material is chosen from: wetting agents, humectants, pH modifiers, surfactants, and combinations thereof.

7. The prosthesis of claim 1, wherein the coating is deposited on the surface of the prosthesis with a coating location accuracy of at least +/−21 microns.

8. The prosthesis of claim 1, wherein the coating is selectively deposited only on areas of minimum strain, and wherein the areas of maximum strain are uncoated.

9. The prosthesis of claim 8, wherein the areas of maximum strain are loops in or bridges between struts of the prosthesis.

10. A stent that can deployed in a patient, the stent comprising:
struts configured in a sinuous geometry;
a coating deposited on at least one surface of linear portions of at least one strut, the coating being deposited by an ink-jet device; and
uncoated surfaces, the uncoated surfaces representing areas of maximum strain of the stent upon deployment in the patient.

11. The stent of claim 10, wherein the thickness of the coating on the stent is conformal or stepped.

12. The stent of claim 11, wherein the conformal coating does not vary in thickness by more than about one micron.

13. The stent of claim 11, wherein the conformal coating is textured, whereby the texture controls the release rate of drug.

14. The stent of claim 10, wherein the areas of maximum strain are loops in or bridges between the struts of the stent.

15. The stent of claim 10, wherein the stent is a bifurcated stent.

16. The stent of claim 10, further comprising a capillary groove disposed in at least one surface of the stens, the groove configured to retain and guide the deposition of at least some portion of the coating.

17. A method of coating a prosthesis, the method comprising the steps of:
depositing a spray of droplets of a material on a prosthesis, wherein the droplets are sprayed by an ink-jet device, and
wherein each of the droplets has a volume of at least about 5 picoliters.

18. The method of claim 17, wherein the prosthesis comprises a capillary groove, and wherein the step of depositing comprises:
depositing the spray of droplets of the material in the capillary groove on the prosthesis.

19. The method of claim 17, wherein the step of depositing comprises:
depositing the spray of droplets of the material with a placement accuracy of at least +/−21 microns.

20. The method of claim 17, wherein the distance between the ink-jet device and the prosthesis is less than or equal to about 1.5 millimeters.

21. The method of claim 17, wherein the volume of each drop is less than or equal to about 200 picoliters, and the velocity of each drop is less than or equal to about 10 meters/second.

22. The method of claim 17, wherein the distance between the ink-jet device and the prosthesis is less than or equal to about 4 millimeters.

23. The method of claim 17, wherein the volume of each drop is less than or equal to about 200 picoliters.

24. The method of claim 17, wherein the velocity of each drop is less than about 10 meters/second.

25. A prosthesis coating system comprising:
an ink-jet device in proximity to the prosthesis, whereby droplets of a material from the ink-jet device can coat at least a portion of the prosthesis; and
an optical micrometer configured to measure material being deposited on the rotating prosthesis by the ink-jet device, wherein the prosthesis is positioned between a transmitter and a receiver in the optical micrometer.

26. The system of claim 25, wherein the ink-jet device is chosen from at least one of: piezo ink-jet, thermal inkjet, acoustic drop ejection, electrostatic inkjet, and combinations thereof.

27. The system of claim 25, further comprising a plurality of ink-jet devices,
wherein a first ink-jet device is positioned to coat a first surface of the prosthesis with a first material, and
wherein a second ink-jet device is positioned to coat a second surface of the prosthesis.

28. The system of claim 27,
wherein the first ink-jet device is positioned to coat an outside surface of the prosthesis with a first material, and
wherein the second ink-jet device is positioned to coat an inside surface of the prosthesis with a second material.
